(19) United States
(12) Patent Application Publication
(10) Pub. No.: US 2019/0284161 A1
(43) Pub. Date: Sep. 19, 2019

(54) 4-((6-(2-(2,4-DIFLUOROPHENYL)-1,1-DIFLUORO-2-HYDROXY-3-(5-MERCAPTO-1H-1,2,4-TRIAZOL-1-YL)PROPYL)PYRIDIN-3-YL)OXY)BENZONITRILE AND PROCESSES OF PREPARATION

(71) Applicant: Dow AgroSciences LLC, Indianapolis, IN (US)

(72) Inventors: Qiang Yang, Zionsville, IN (US); Kaitlyn Gray, Indianapolis, IN (US); Nicholas R. Babij, Indianapolis, IN (US); Xiaoyong Li, Midland, MI (US); Yan Hao, Zionsville, IN (US)

(21) Appl. No.: 16/462,207

(22) PCT Filed: Nov. 17, 2017
(86) PCT No.: PCT/US2017/062129

§ 371 Date: May 17, 2019

Related U.S. Application Data
(60) Provisional application No. 62/423,871, filed on Nov. 18, 2016.

(51) Int. Cl. C07D 401/06

(52) U.S. Cl. CPC C07D 401/06

(57) ABSTRACT

Provided herein is a process for the preparation of 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile.

### CROSS-REFERENCE TO RELATED APPLICATIONS

The present application claims priority under 35 U.S.C. § 119(e) to U.S. provisional patent application, U.S. Ser. No. 62/423,871, filed Nov. 18, 2016, the entire contents of which is incorporated herein by reference.

### FIELD

Provided herein is 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile and processes of preparation.

### BACKGROUND

U.S. Patent Application Ser. No. 62/163,106 describes inter alia certain metalloenzyme inhibitor compounds and their use as fungicides. The disclosure of this application is expressly incorporated by reference herein. This patent application describes various routes to generate metalloenzyme inhibiting fungicides. It may be advantageous to provide more direct and efficient methods for the preparation of metalloenzyme inhibiting fungicides and related compounds, e.g., by the use of reagents and/or chemical intermediates which provide improved time and cost efficiency.

### SUMMARY OF THE DISCLOSURE

Provided herein is the compound 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I) and processes for its preparation. In one embodiment, provided herein, is a process for the preparation of the compound of the Formula I:

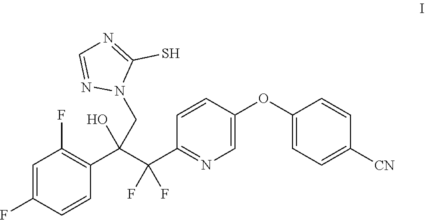 [SMILES: N#Cc1ccc(Oc2ccc(C(F)(F)C(O)(Cn3ncnc3S)c3ccc(F)cc3F)nc2)cc1]

which comprises contacting a compound of Formula II with a sulfur reagent.

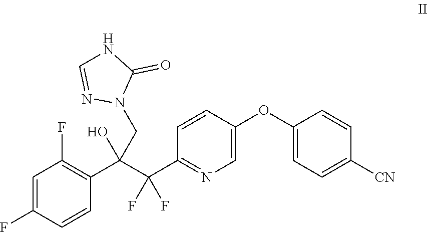 [SMILES: N#Cc1ccc(Oc2ccc(C(F)(F)C(O)(Cn3nc[nH]c3=O)c3ccc(F)cc3F)nc2)cc1]

In another embodiment, the compound of Formula II may be prepared by contacting a compound of Formula III with an organic carbamate, 1,1-dimethoxy-N,N-dimethylmethanamine and an acid.

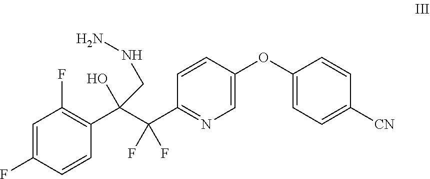 [SMILES: N#Cc1ccc(Oc2ccc(C(F)(F)C(O)(CNN)c3ccc(F)cc3F)nc2)cc1]

Another aspect of the present disclosure is the novel intermediate produced in the present process, viz., a compound consisting of:

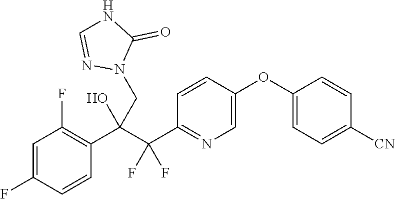 [SMILES: N#Cc1ccc(Oc2ccc(C(F)(F)C(O)(Cn3nc[nH]c3=O)c3ccc(F)cc3F)nc2)cc1]

The term “halogen” or “halo” refers to one or more halogen atoms, defined as F, Cl, Br, and I.

The term “organometallic” refers to an organic compound containing a metal, especially a compound in which a metal atom is bonded directly to a carbon atom.

Room temperature (RT) is defined herein as about 20° C. to about 25° C.

Throughout the disclosure, references to the compounds of Formula I-III are read as also including optical isomers and salts. Specifically, when compounds of Formula I-III contain a chiral carbon, it is understood that such compounds include optical isomers and racemates thereof. Exemplary salts may include: hydrochloride salts, hydrobromide salts, hydroiodide salts, and the like.

Certain compounds disclosed in this document can exist as one or more isomers. It will be appreciated by those skilled in the art that one isomer may be more active than the others. The structures disclosed in the present disclosure are drawn in only one geometric form for clarity, but are intended to represent all geometric and tautomeric forms of the molecule. For example, the chemical structures of Formulas I and Ia are tautomeric forms of the same molecule.

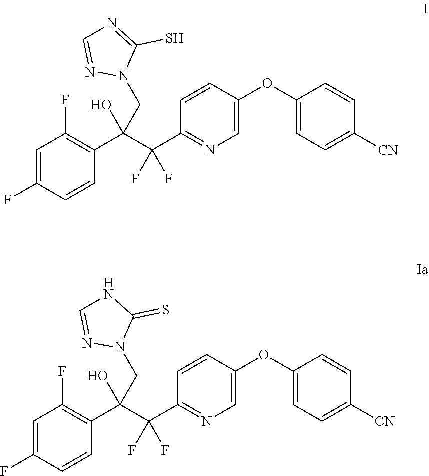 [SMILES: N#Cc1ccc(Oc2ccc(C(F)(F)C(O)(Cn3ncnc3S)c3ccc(F)cc3F)nc2)cc1 ; N#Cc1ccc(Oc2ccc(C(F)(F)C(O)(Cn3nc[nH]c3=S)c3ccc(F)cc3F)nc2)cc1]

The embodiments described above are intended merely to be exemplary, and those skilled in the art will recognize, or will be able to ascertain using no more than routine experimentation, numerous equivalents of specific processes, materials and procedures. All such equivalents are considered to be within the scope of the invention and are encompassed by the appended claims.

### DETAILED DESCRIPTION

4-((6-(2-(2,4-Difluorophenyl)-1,1-difluoro-2-hydroxy-3-(5-mercapto-1H-1,2,4-triazol-1-yl)propyl)pyridin-3-yl)oxy)benzonitrile (I) is provided herein and may be prepared from 4-((6-(2-(2,4-difluorophenyl)-1,1-difluoro-2-hydroxy-3-(5-oxo-4,5-dihydro-1H-1,2,4-triazol-1-yl)propyl)pyridin-3-yl)oxy)benzonitrile (II) as shown in Example 1.

### Example 1: Preparation of 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I)

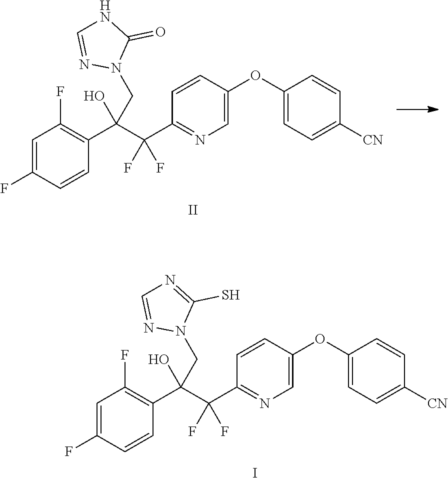

To a 100-mL round bottom flask was charged 4-((6-(2-(2,4-difluorophenyl)-1,1-difluoro-2-hydroxy-3-(5-oxo-4,5-dihydro-1H-1,2,4-triazol-1-yl)propyl)pyridin-3-yl)oxy)benzonitrile (II; 2 g, 4.12 mmol), phosphorus pentasulfide (0.916 g, 4.12 mmol), and acetonitrile (20 mL). The reaction was heated at 75° C. for 4 h, at which point HPLC analysis indicated that the reaction was complete. The reaction mixture was cooled to 20° C. and concentrated to dryness. The residue was purified by silica gel column chromatography (80 g silica, 5 column volumes 0-50% EtOAc/hexanes, hold for 10 column volumes). The fractions containing the pure product were combined and concentrated to afford the desired product (I) as a pale yellow solid (1.6 g, 77% yield). ¹H NMR (400 MHz, DMSO-d₆) δ 13.59 (s, 1H), 8.46 (d, J=2.7 Hz, 1H), 8.18 (s, 1H), 7.91 (d, J=8.3 Hz, 2H), 7.71 (dd, J=8.7, 2.7 Hz, 1H), 7.63 (d, J=8.7 Hz, 1H), 7.37 (q, J=8.3 Hz, 1H), 7.18 (dd, J=28.1, 9.5 Hz, 3H), 6.95 (t, J=7.4 Hz, 1H), 6.42 (s, 1H), 5.20-4.92 (m, 2H). ESIMS m/z 502.0 [(M+H)⁺].

Sulfur reagents for use in this process may include, but are not limited to, phosphorus pentasulfide (P₂5₅), Lawesson's reagent, and hydrogen sulfide or derivatives thereof.

Suitable solvents for use in this process step may include, but are not limited to, acetonitrile (MeCN), ethyl acetate, toluene, THF, dioxane, and ethanol.

This process step may be conducted at temperatures from about 25° C. to about 150° C., or from about 50° C. to about 100° C.

4-((6-(2-(2,4-Difluorophenyl)-1,1-difluoro-2-hydroxy-3-(5-oxo-4,5-dihydro-1H-1,2,4-triazol-1-yl)propyl)pyridin-3-yl)oxy)benzonitrile (II) may be prepared from 4-((6-(2-(2,4-difluorophenyl)-1,1-difluoro-3-hydrazino-2-hydroxypropyl)pyridin-3-yl)oxy)benzonitrile (III) as shown in Example 2.

### Example 2: Preparation of 4-((6-(2-(2,4-difluorophenyl)-1,1-difluoro-2-hydroxy-3-(5-oxo-4,5-dihydro-1H-1,2,4-triazol-1-yl)propyl)pyridin-3-yl)oxy)benzonitrile (II)

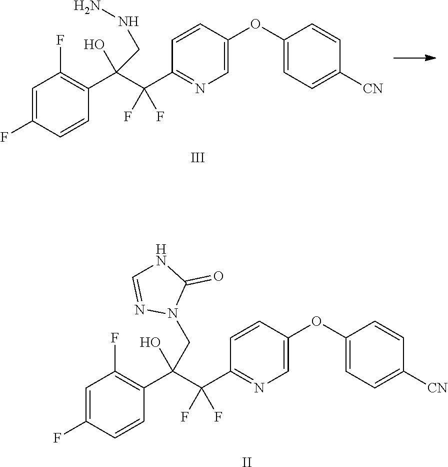

To a 4-neck, 250-mL round bottom flask was charged ethyl carbamate (2.164 g, 24.28 mmol), 1,1-dimethoxy-N,N-dimethylmethanamine (3.23 mL, 24.28 mmol), and toluene (21 mL). The reaction was heated at 85° C. for 2 h. 4-((6-(2-(2,4-Difluorophenyl)-1,1-difluoro-3-hydrazino-2-hydroxypropyl)pyridin-3-yl)oxy)benzonitrile (III) (7.0 g, 16.19 mmol) and acetic acid (6.94 mL, 121 mmol) were added and the reaction was heated at 85° C. for 2 h. The reaction mixture was cooled to 20° C. and quenched with saturated sodium bicarbonate solution to pH 7-8. The mixture was extracted with EtOAc (50 mL) and the organic layer was concentrated to afford a yellow oil, which was purified by silica gel column chromatography (330 g silica, 2% MeOH/DCM). The fractions containing the pure product were concentrated to afford a white foam (4.5 g, 57% yield). ¹H NMR (400 MHz, CDCl₃) δ 11.64 (s, 1H), 8.36 (d, J=2.7 Hz, 1H), 7.78-7.58 (m, 2H), 7.58-7.41 (m, 2H), 7.37 (d, J=12.4 Hz, 2H), 7.13-6.98 (m, 2H), 6.73 (ddt, J=10.4, 7.0, 2.7 Hz, 2H), 6.48 (s, 1H), 5.12 (d, J=15.0 Hz, 1H), 4.67-4.45 (m, 1H). ¹³C NMR (101 MHz, CDCl₃) δ 163.29 (dd, J=250.9, 12.1 Hz), 159.98 (dd, J=250.2, 11.9 Hz), 159.62, 156.31, 152.94, 148.07 (t, J=28.3 Hz), 140.61, 134.54, 134.47, 131.94 (dd, J=9.6, 5.0 Hz), 126.88, 123.86 (t, J=4.3 Hz), 119.87 (dd, J=12.8, 3.5 Hz), 118.89, 118.20, 110.96 (dd, J=20.8, 2.9 Hz), 110.87, 107.92, 104.29 (dd, J=29.0, 25.6 Hz), 79.26, 79.22, 78.96 (td, J=28.5, 4.4 Hz), 50.17. ESIMS m/z 486.1 [(M+H)⁺].

Suitable organic carbamates for use in this process step may include alkyl carbamates and aryl carbamates of the following formula:

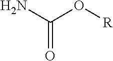

wherein R is a C₁-C₆ alkyl group or an aryl group such as, for example, a substituted or an unsubstituted phenyl group.

Suitable acids for use in this process step may include, but are not limited to, acetic acid, formic acid, trifluoroacetic acid, tosylsulfonic acid, boron trifluoride etherate, hydrochloric acid, and hydrobromic acid.

Suitable solvents for use in this process step may include, but are not limited to, toluene, a xylene, ethyl acetate, THF, acetonitrile, and mixtures thereof.

This process step may involve the use of a pre-heating step for a mixture of the organic carbamate, the 1,1-dimethoxy-N,N-dimethylmethanamine and the solvent prior to addition of the compound of Formula III and the acid to the process. This pre-heating step may be conducted for about 1 to about 5 hours at temperatures from about 50° C. to about 150° C., or from about 50° C. to about 100° C.

After completion of the pre-heating step and addition of compound III and the acid, the process may be conducted at temperatures from about 25° C. to about 150° C., or from about 50° C. to about 100° C.

In some embodiments an orthoformate can be used in place of the 1,1-dimethoxy-N,N-dimethylmethanamine for this type of transformation as described in Angew Chem. 1968, 918.

### Example 3: Preparation of 4-((6-(2-(2,4-difluorophenyl)-1,1-difluoro-3-hydrazino-2-hydroxypropyl)pyridin-3-yl)oxy)benzonitrile (III)

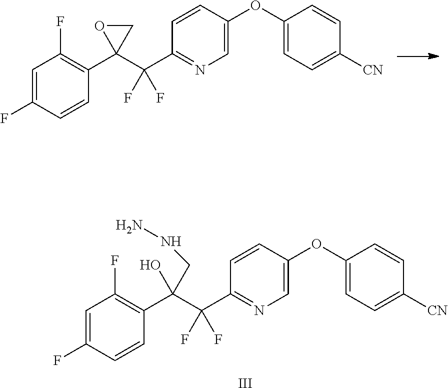

To a slurry of 4-((6-(2-(2,4-difluorophenyl)oxiran-2-yl)difluoromethyl)pyridin-3-yl)oxy)benzonitrile (5 g, 12.49 mmol) in ethanol (50.0 ml) was added anhydrous hydrazine (1.0 ml, 31.2 mmol, 2.5 eq) and the reaction was heated to 60° C. for 4 h, at which point the starting epoxide was completely consumed (monitored by HPLC). The reaction was allowed to cool to room temperature overnight during which time a white precipitate formed. The solids were isolated by filtration and rinsed with ethanol (15 mL) followed by MTBE (15 mL). The solid was dried under vacuum giving 4-((6-(2-(2,4-difluorophenyl)-1,1-difluoro-3-hydrazino-2-hydroxypropyl)pyridin-3-yl)oxy)benzonitrile (III) as an off white solid (4.4 g, about 85% purity, 8.65 mmol, 69.3% corrected yield). ¹H NMR (400 MHz, CDCl₃) δ 8.38 (d, J=2.6 Hz, 1H), 7.70-7.65 (m, 2H), 7.64-7.55 (m, 1H), 7.45 (d, J=8.6 Hz, 1H), 7.36 (dd, J=8.7, 2.7 Hz, 1H), 7.08-7.02 (m, 2H), 6.85-6.71 (m, 2H), 3.76 (d, J=13.4 Hz, 1H), 3.62 (dd, J=13.4, 2.3 Hz, 1H). ¹⁹F NMR (376 MHz, CDCl₃) δ −105.40 (ddd, J=21.5, 16.2, 8.8 Hz), −109.57 (d, J=21.5 Hz), −109.77 (d, J=16.1 Hz), −110.58 (d, J=8.8 Hz). ESI MS m/z 433.1 [(M+H)⁺].

What is claimed is:

1. A method of making a compound of Formula I comprising: 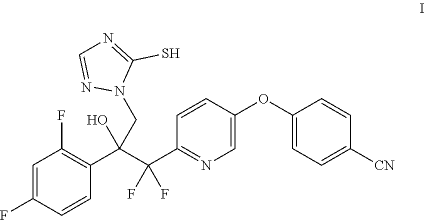 [SMILES: N#Cc1ccc(Oc2ccc(C(F)(F)C(O)(Cn3ncnc3S)c3ccc(F)cc3F)nc2)cc1]
contacting a compound of Formula II 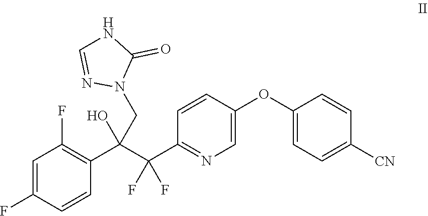 [SMILES: N#Cc1ccc(Oc2ccc(C(F)(F)C(O)(Cn3nc[nH]c3=O)c3ccc(F)cc3F)nc2)cc1]
with a sulfur reagent.

2. The method of claim 1 wherein the sulfur reagent may be selected from the group including phosphorus pentasulfide, Lawesson's reagent, and hydrogen sulfide, or derivatives of hydrogen sulfide.

3. The method of claim 1 wherein the sulfur reagent is phosphorus pentasulfide.

4. The method of claim 1 further comprising a solvent selected from the group including acetonitrile, ethyl acetate, toluene, THF, dioxane, ethanol, and mixtures thereof.

5. The method of claim 1 wherein the contacting is carried out between about 25° C. and about 150° C.

6. The method of claim 1 wherein the contacting is carried out between about 50° C. and about 100° C.

7. The method of claim 1, further comprising the step of contacting a compound of Formula III 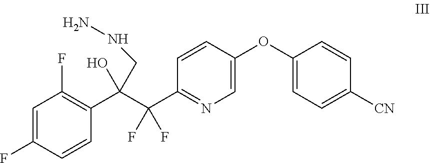 [SMILES: N#Cc1ccc(Oc2ccc(C(F)(F)C(O)(CNN)c3ccc(F)cc3F)nc2)cc1]
with an organic carbamate, 1,1-dimethoxy-N,N-dimethylmethanamine, and an acid to produce the compound of Formula II.

8. The method of claim 7 further comprising a pre-heating step whereby a mixture of the organic carbamate, the 1,1-dimethoxy-N,N-dimethylmethanamine and a solvent are combined and pre-heated prior to addition of the acid and the compound of Formula III.

9. The method of claim 7 further comprising a solvent selected from the group including toluene, a xylene, ethyl acetate, THF, acetonitrile, and mixtures thereof.

10. The method of claim 7 wherein the acid is acetic acid.

11. The method of claim 7 wherein the alkyl carbamate is ethyl carbamate.

12. The method of claim 7 wherein the contacting is carried out from about 25° C. to about 150° C.

13. The method of claim 7 wherein the contacting is carried out from about 50° C. to about 100° C.

14. The method of claim 8 wherein the pre-heating is conducted at about 50° C. to about 150° C.

15. The method of claim 8 wherein the pre-heating is conducted at about 50° C. to about 100° C.

16. A compound consisting of: 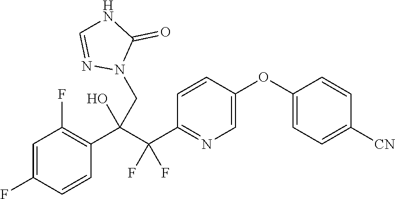 [SMILES: N#Cc1ccc(Oc2ccc(C(F)(F)C(O)(Cn3nc[nH]c3=O)c3ccc(F)cc3F)nc2)cc1]
